(19) United States
(12) Patent Application Publication
(10) Pub. No.: US 2008/0194997 A1
(43) Pub. Date: Aug. 14, 2008

(54) System and method for diagnosing and treating patellar maltracking and malalignment

(75) Inventor: Li-Qun Zhang, Wilmette, IL (US)

Correspondence Address: DRINKER BIDDLE & REATH LLP;ATTN: PATENT DOCKET DEPT., 191 N. WACKER DRIVE, SUITE 3700, CHICAGO, IL, 60606, US

(73) Assignee: Rehabilitation Institute of Chicago, Chicago, IL (US)

(21) Appl. No.: 11/704,557

(22) Filed: Feb. 8, 2007

(51) Int. Cl. A61B 5/11; A61F 5/00

(52) U.S. Cl. 600/595; 602/26; 602/7

(57) ABSTRACT

A system and method are provided for in vivo, noninvasive diagnosis of patellar and scapular malalignment and maltracking. The patellar system and method include a patella-engaging apparatus and a motion analysis system. The patella-engaging apparatus includes a member that has a custom-contoured posterior surface that is configured to engage an anterior surface of a subject's patella. A plurality of markers is coupled with the anterior surface of the member and the motion analysis system optically tracks the plurality of markers. The system and method may optionally include a second portion such as a goniometer that tracks relative movement of the tibial and femoral portions of the subject's leg proximate to the patella. The member that has the custom-contoured posterior surface may be employed in a method and system for treating malalignment and maltracking, and a method for evaluating the effectiveness of treatment of the same.

### STATEMENT REGARDING FEDERALLY SPONSORED RESEARCH AND DEVELOPMENT

This invention was made in part with Government support under Grant Number R01 AR45634 awarded by the National Institutes of Health. The Government may have certain rights in this invention.

### FIELD OF THE INVENTION

This invention pertains generally to medical diagnostic and treatment systems and methods. More particularly, this invention relates to an in vivo, noninvasive system and method for diagnosing and treating patellar maltracking and malalignment.

### BACKGROUND OF THE INVENTION

Patellofemoral pain (PFP) syndrome is one of the most commonly observed physical abnormalities involving the knee, with reported incidence rates greater than 25% among athletes. Current literature suggests that the etiology of PFP is multifactorial, although it is commonly accepted that abnormal patellar tracking and patellar malalignment are closely related to PFP syndrome. However, accurate clinical assessment of patellar maltracking and malalignment is difficult because no practical, cost effective methods and systems exist to accurately and quantitatively evaluate patellar motion and alignment in vivo and noninvasively.

Historically, patellar alignment and tracking are generally assessed clinically by visually inspecting the orientation and position of the patella relative to the femur during manual manipulation under static and dynamic conditions. Such clinical assessment is convenient but lacks accuracy, repeatability and reliability. The orientation of the patella relative to the femur can also be examined at a fixed knee flexion angle with axial radiographs. However, axial radiographs may not give an accurate assessment because such radiographs are typically obtained statically in a flexed knee in which the malaligned patella may be centered and seated in the trochlear groove due to tightening of surrounding soft tissues and muscles. Dynamic patellar tracking and static patellar alignment have been assessed more accurately by computed tomography (CT) and magnetic resonance imaging (MRI) at different knee flexion angles. Although such scanning methods are useful to quantitatively measure static knee joint structure and dynamic patellar tracking, such methods are not cost effective. Furthermore, limb movement during scanning will affect the accuracy of scan results.

In an attempt to overcome the disadvantages of the forgoing methods, in vivo patellar tracking procedures have been performed. However, these in vivo procedures were invasive and involved inserting intracortical pins into the patella and femur in both healthy and PFP subjects. Such invasive procedures are disadvantageous from the standpoints of potential subsequent complications (e.g., infection, etc.) and lack of repeatability due to difficulty in removing and re-inserting the intracortical pins in their previous locations, for example subsequent to treatment of the diagnosed malalignment or maltracking. Moreover, in vivo, invasive procedures can not be efficiently and expeditiously applied to a large number of subjects, for example test and control subjects of an experiment, due to the cost and time to properly insert, remove and re-insert intracortical pins.

In view of the foregoing, it can be appreciated that there is a need to evaluate patellar tracking in vivo and noninvasively. However, accurate in vivo, noninvasive tracking of the patella has been difficult due to a number of factors:

(1) The movement of the patella is true three-dimensional (3D) movement—As shown in FIG. 1, when the knee is fully extended the patella P “floats” above the trochlear groove TG of the femur F. Then, as the knee is flexed, the patella P follows the contour of the trochlear groove, by flexing, shifting, and rotating in all three dimensions.

(2) Small amplitude motions—Referring now to FIG. 2, which illustrates a patellofemoral joint-coordinate system (JCS), although the primary motion of the patella P is relative to the flexion-extension axis (X_{f}) of the femur F, other motions thereof are believed to be etiological in PFP. In particular, rotations of the patella P relative to the two axes Zₚ, Yₚ (medial-lateral rotation and medial-lateral tilt), and translation/shifting of the patella P along the medial-lateral axis (Xₚ) are believed to be important factors. Since these medial-lateral rotation, medial-lateral tilt and medial-lateral shifting motions of the patella are generally small amplitude motions, the effects of error (e.g., measurement error) are proportionately larger.

(3) Sliding action under the skin—The patella is a sesamoid bone that develops on the posterior side of the quadriceps tendon. Therefore, the anterior side of the patella is essentially encapsulated within the quadriceps tendon. As the knee flexes, the patella moves distally, sliding beneath the skin. Thus, any markers placed on the skin when the knee is in extension would be significantly proximal to the patella when the knee is flexed. Furthermore, as best evidenced during squatting and walking, the skin above the patella and the patella itself do not move synchronously, i.e., at the same time and in the same pattern and direction. Therefore, markers affixed to the skin, such as the ones commonly used for tracking the femur and tibia, are particularly ill-suited for tracking the patella.

One noninvasive system and method for in vivo measurement of patellar movement that overcomes a number of shortcomings and difficulties in the forgoing-described diagnostic methods is disclosed in the article “In vivo and noninvasive six degrees of freedom patellar tracking during voluntary knee movement,” by Lin et al. which was published in Clinical Biomechanics 18 (2003) on pages 401-409, the disclosure of which is incorporated herein by reference in its entirety.

In the Lin et al. article, patellar tracking in three-dimensions was facilitated through the use of a clamp member that is mounted to the patella. Movement of the patella is tracked and analyzed in six degrees of freedom according to the joint coordinate system (JCS) shown in FIG. 2 wherein patellofemoral and tibiofemoral movements are described by three X-Y-Z coordinate systems (axes labeled with subscripts f, t and p) relative to the femur (F), tibia (T) and patella (P). As shown in FIGS. 3a-3c, the clamp member 100 included: a top plate 110; a medial plate 120 with medial patella-engaging members 122 (e.g., Teflon strips) and medial adjustment screws 124; a lateral plate 130 with lateral patella-engaging members 132 (e.g., Teflon strips) and lateral adjustment screws 134; and a plurality of markers 140 (e.g., reflective markers, high-visibility LEDs, infrared LEDs, etc.) configured on the top plate 110. The clamp member 100 is removably coupled with the patella P by configuring it on the knee of a subject over the patella P and adjusting the medial and lateral adjustment screws 124, 134 so that the respective medial and lateral patella-engaging members 122, 132 are moved to grasp medial and lateral edges of the patella P. In this way, the clamp member 100 moves together with the patella P so that the plurality of markers 140 can be tracked using still or moving picture camera(s), a vision system, optical motion analysis system (e.g., OptoTrak® 3020 available from Northern Digital, Inc. of Waterloo, Canada) or the like to analyze the movement of the patella P.

While the Lin et al. system provides a helpful, accurate and consistent method for in vivo, noninvasive diagnosis of patellar maltracking and malalignment, it has limitations in that the clamp member 100 can only be reliably mounted on the edges of the patella for accurate tracking of the patella through a small range of knee flexion angles (approximately between full extension and 20° flexion) due to soft tissue loading. Because evaluation of patellar tracking through a wide range of knee motion (e.g., during functional activities) may provide key diagnostic information to aid in classification and treatment of PFP, a new method and system for in vivo and noninvasive evaluation and treatment of patellar tracking would be an important improvement in the art.

### BRIEF SUMMARY OF THE INVENTION

In a first aspect, a system and method are provided for in vivo, noninvasive diagnosis of patellar malalignment and maltracking. An example diagnostic system comprises a motion analysis system and a patella-engaging apparatus including a patella-engaging member that is contoured to the patella of a subject. The patella-engaging member includes a custom-formed posterior surface that is configured to engage the anterior surface of a subject's patella. Because the skin in front of (i.e., anterior) the patella and the patella itself do not move synchronously or at precisely the same time, pattern or direction (as is best evidenced during squatting and walking activities), a coupling such as, for example an elastic band or wrap, is provided to press an anterior surface of the patella-engaging member so that the custom-formed posterior surface is urged into engagement with the subject's patella. By properly tightening (i.e., not too tight or too loose) the coupling, the skin of the subject's knee in the patella region becomes clamped between the patella and the patella-engaging member so that the patella-engaging member synchronously moves with the subject's patella, but does not interfere with its movement.

In the example diagnostic system, the motion analysis system comprises a first portion that includes an optical system and a plurality of markers that is attached to the anterior surface of the patella-engaging member. The plurality of markers is configured to be tracked by the optical system. In some embodiments the coupling may be configured to substantially cover or wrap around the subject's knee and may include at least one aperture through which the plurality of markers is visible. The motion analysis system may also include a second portion such as a goniometer that tracks relative movement of the tibial and femoral portions of the subject's leg proximate to the patella. In other example systems, the foregoing-described patella-engaging apparatus may be adapted for in vivo, noninvasive diagnosis of malalignment and maltracking of other human or animal anatomical joints such as, for example, the scapula.

In the first aspect, an example method for in vivo, noninvasive diagnosis of patellar malalignment and maltracking comprises the steps of: configuring a patella-engaging member to have a posterior surface that is custom-contoured to the knee of a subject in the patella region; affixing a plurality of markers on an anterior surface of the patella-engaging member; coupling the patella-engaging member with the knee of the subject; and tracking movement of the plurality of markers in three dimensions during a functional activity being performed by the subject. The step of coupling the patella-engaging member to the knee of the subject may include the steps of: orienting the patella-engaging member over the patella of the subject; and wrapping an elastic member about the patella-engaging member and the knee of the subject so that the patella-engaging member moves synchronously together with the patella, but without interfering with its movement. The method may also include the step of tracking relative movement of the tibial and femoral portions of the subject's leg proximate to the patella.

In a second aspect, a system and method are provided for nonoperative treatment of patellar malalignment and maltracking. Because the foregoing-mentioned patella-engaging member is custom-fit to a subject's knee in the patella region, it can be used to engage the patella for helping to move the patella into a desired position and orientation (e.g., aligned with the trochlear groove TG shown in FIG. 1) to correct the malalignment and maltracking. An example treatment system comprises: a patella-engaging member including a custom-formed posterior surface that is configured to engage the anterior surface of a subject's patella for moving the subject's patella to a realigned orientation; and an alignment-maintaining member that couples with the patella-engaging member for supporting the subject's patella in the realigned orientation. In some embodiments the alignment-maintaining member may comprise tape, however, in other embodiments the alignment-maintaining member may comprise an orthotic device such as, for example, a knee brace. The patella-engaging member may be permanently or removably attached to a patellar portion of the orthotic device. In some embodiments of the system, the posterior surface of the patella-engaging member may include a layer of padding to improve the comfort of the subject. Furthermore, in another example system, the foregoing-described patella-engaging apparatus may be adapted for nonoperative treatment of malalignment and maltracking of other human or animal anatomical joints such as, for example, the scapula.

In the second aspect, one example treatment method comprises the steps of: configuring a patella-engaging member to have a posterior surface that is custom-contoured to the knee of a subject in the patella region; engaging the patella-engaging member with the subject's patella; moving the patella-engaging member to position the patella in a realigned position and orientation; and taping the patella-engaging member to the knee of the subject to maintain the patella in the realigned position and orientation. Another example treatment method comprises the steps of: configuring a patella-engaging member to have a posterior surface that is custom-contoured to the knee of a subject in the patella region; attaching the patella-engaging member to a patellar portion of an orthotic device; and fitting the orthotic device to the knee of the subject so that the patella-engaging member moves the patella to a realigned position and orientation and maintains the patella in the realigned position and orientation.

Because the foregoing-mentioned patella-engaging member is custom-fit to a subject's patella and, therefore, is useful for providing accurate and repeatable diagnosis, in yet another aspect a method for evaluating the effectiveness of treatment of patellar malalignment and maltracking is provided. An example method comprises the steps of: a) determining a pre-treatment amount of patellar malalignment and maltracking in vivo and noninvasively; b) treating the determined malalignment and maltracking; c) determining a post-treatment amount of patellar malalignment and maltracking in vivo and noninvasively subsequent to the treating step; and d) comparing the pre-treatment and post-treatment amounts of patellar malalignment and maltracking. Determining step a) may include the steps of custom-configuring a patella-engaging member relative to a knee of a subject in the patella region; configuring a plurality of markers on an anterior side of the patella-engaging member; coupling the patella-engaging member to the knee of the subject; and tracking movement of the plurality of markers in three dimensions during a functional activity being performed by the subject.

The treating step b) may include one or more steps including, but not limited to: strengthening muscle(s) of the subject's leg; performing an appropriate surgical procedure on the subject's leg to improve functionality of the patella; and nonoperative treatment such as taping the subject's knee and/or fitting the subject with one or more orthotic devices such as knee braces, shoe inserts, etc. In some instances the patella-engaging member may be used in the treating step b) in combination with tape or an orthotic device by, for example, removing the plurality of markers from the patella-engaging member. If the plurality of markers were removed for employing the patella-engaging member during the treating step b), the markers may be subsequently reattached to the patella-engaging member during the determining step c).

### BRIEF DESCRIPTION OF THE DRAWINGS

FIG. 1 partially illustrates the anatomy of a patella relative to a distal femur portion;

FIG. 2 illustrates a patellofemoral joint coordinate system (JCS) that is employed herein for tracking and analyzing patellar movement in six degrees of freedom;

FIGS. 3a-3c illustrate views of a conventional patella-engaging member;

FIG. 4 illustrates an example system for in vivo, noninvasive diagnosis of patellar malalignment and maltracking;

FIG. 5 illustrates an example patella-engaging apparatus;

FIG. 6 illustrates a side view of an example patella-engaging member of the patella-engaging apparatus shown in FIG. 5;

FIG. 7 illustrates a front view of the example patella-engaging member of FIG. 6; and

FIGS. 8a and 8b graphically illustrate results from the example motion analysis system of FIG. 7, the graphs comparing patellar tracking of healthy and PFP test subjects during functional activities.

### DETAILED DESCRIPTION

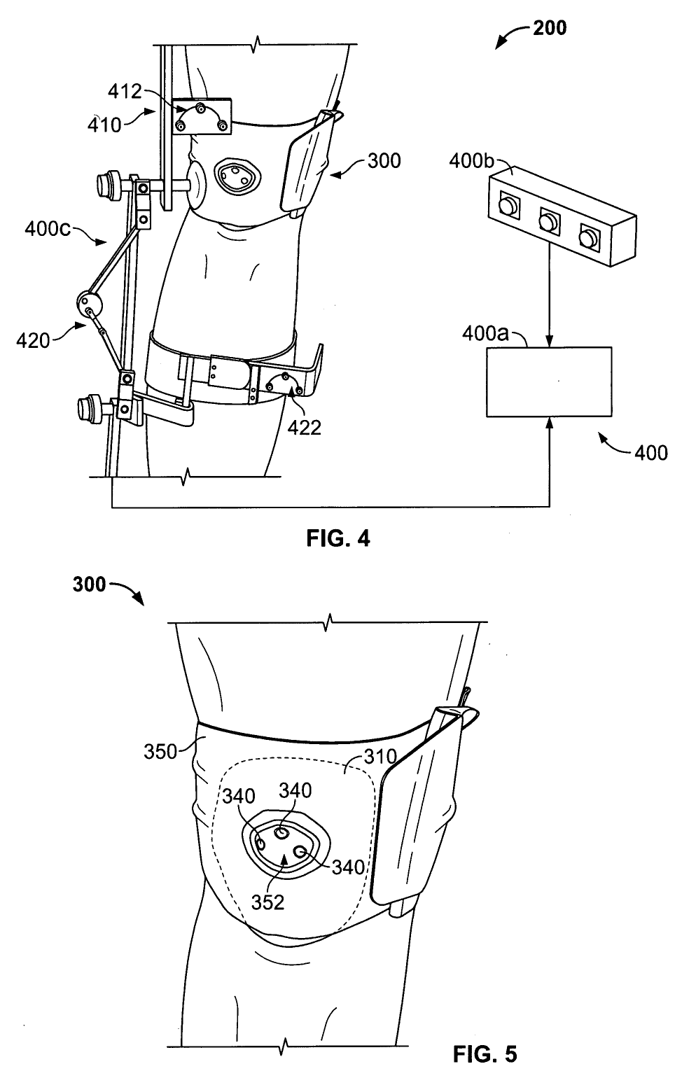

Referring now to the Figures, an improved system and method is provided for in vivo, noninvasive diagnosis and treatment of patellar maltracking and malalignment. Although the improved system and method is described in the context of being particularly useful for diagnosing and treating patellar maltracking and malalignment, it should be appreciated that the described system and method may be adapted and employed for diagnosing and treating maltracking and malalignment in other human and animal joints as well, for example the scapula/shoulder blade. As shown in FIG. 4, the improved system 200 for in vivo, noninvasive diagnosis of patellar maltracking and malalignment comprises a patella-engaging apparatus 300 and a motion analysis system 400. As shown, the motion analysis system 400 includes a computer 400a, a first measuring portion 400b that optically tracks movement of the patella-engaging apparatus 300 during, for example a functional activity such as walking, squatting, etc. being performed by the subject, and an optional second measuring portion 400c that is configured to measure relative movement of the femoral and tibial portions of the subject's leg.

The computer 400a includes a communications portion for interfacing with and receiving communications (e.g, signals, data, etc.) from the first and second measuring portions 400b, 400c, and a processor for analyzing communications (e.g, signals, data, etc.) received from the first and second measuring portions 400b, 400c. In some embodiments the computer 400a may include and execute a software program or algorithm that facilitates gait analysis or motion analysis. The first and second measuring portions 400b, 400c may communicate with the computer 400a using various protocols known in the art (e.g., serial or parallel communication). Moreover, the first and second measuring portions 400b, 400c may communicate with the computer 400a wirelessly or via wires. The computer 400a may be any suitable processing device known in the art such as a mainframe, a laptop or desktop personal computer (PC) with an operating system such as Windows, Mac OS, Linux, Unix, etc., or the like. Furthermore, the computer 400a may include and be configured to execute one or more software applications known in the art for motion analysis, gait analysis and data analysis so that the computer 400a is operative to process communications from the first and second measuring portions 400b, 400c and determine patellar maltracking and malalignment. Moreover, the computer 400a may include various peripherals known in the art such as user interface devices for example, a display, keyboard, mouse, printer, etc.

The first measuring portion 400b is configured to optically track movement of the patella-engaging apparatus 300 relative to motions of the subject's knee and/or leg. More specifically, the first measuring portion 400b tracks movement of markers 340 (FIG. 5) that are coupled with or attached directly or indirectly (e.g., by a Y-shaped frame known as a triad) on the patella-engaging apparatus 300, which will be described in greater detail hereinafter with reference to FIGS. 5-7. In the system 200, although the markers 340 are shown and described herein to be coupled with, attached to, configured on or otherwise associated with the patella-engaging apparatus 300, the patella-engaging apparatus 300 is not limited to include the markers 340 because, as can be appreciated, the markers 340 may be considered as a portion of the first measuring portion 400b.

The first measuring portion 400b includes optical devices such as lenses, imaging devices (e.g., complementary metal oxide semiconductor (CMOS) image sensor, charge coupled device (CCD), etc.), and the like for dynamically focusing and capturing images of the subject's knee and/or leg during a functional activity, such as, for example walking, squatting, etc., which is being performed by the subject. The first measuring portion 400b may include various image-capturing devices and/or systems known in the art. In one embodiment, the first measuring portion 400b may include the OptoTrak® 3020, which is available from Northern Digital, Inc. of Waterloo, Canada. However, in other embodiments, the first measuring portion 400b may include a vision system, one or more still or moving picture cameras (e.g., digital still camera, digital video camcorder, etc.) configured to provide a sequence or stream of images relative to two or more aspects (i.e., visual angles or perspectives), or the like.

The second measuring portion 400c, which is optional in some embodiments of the system 200, is configured to further track or measure movement of portions of the subject's leg relative to the patella-engaging apparatus 300. As can be appreciated from FIG. 4, the second measuring portion 400c is configured to measure relative movement of the subject's thigh/femur and shin/tibia with reference to the subject's knee. As shown, the second measuring portion includes a femoral portion 410 that couples with the thigh of the subject, and a tibial portion 420 that couples with the shin of the subject. The femoral and tibial portion 410, 420 may be coupled together at a rotary encoder or the like for relative rotation so that the second measuring portion 400c may be employed as a measuring device such as a goniometer for measuring angular rotation/motion of the tibia relative to the femur during a functional activity, such as, for example walking, squatting, etc., which is being performed by the subject. Furthermore, as shown in FIG. 4, the second measuring portion 400c may include a plurality of markers 412, 422 so that the first measuring portion 400b may optically track movement of the thigh and shin simultaneously with movement of the knee. The plurality of markers on the second measuring portion 400c may be configured so that a first plurality of markers 412 are coupled with the femoral portion 410 and a second plurality of markers 422 are coupled with the tibial portion 420.

Referring now to FIGS., 5-7, the patella-engaging apparatus 300 is described. As shown in FIG. 5, the patella-engaging apparatus 300 includes a patella-engaging member 310 and a coupling 350 that releasably secures the patella-engaging member 310 to the subject's patella. As previously mentioned, although the markers 340 are shown and described herein to be coupled with, attached to, configured on or otherwise associated with the patella-engaging apparatus 300, the patella-engaging apparatus 300 is not limited to include the markers 340 because, as can be appreciated, the markers 340 may be considered as a portion of the first measuring portion 400b. As shown, the coupling 350 may be a band or strap that substantially covers or wraps around the subject's knee and the patella-engaging member 310. As shown, the coupling 350 is configured to be wider (i.e., taller) than the vertical dimension of the patella-engaging member 310 so that the coupling 350 may be tightened properly (i.e., not too tight or too loose) to provide sufficient pressure against an anterior surface of the patella-engaging member 310, thereby causing the skin of the subject's knee in the patella region to become clamped between the patella and the patella-engaging member 310 so that the patella-engaging member 310 synchronously moves with the subject's patella, but does not interfere with its movement. Furthermore, when tightened properly, the coupling 350 does not cause the subject undue discomfort due to compression of the subject's skin.

As further shown, the coupling 350 includes at least one aperture 352 through which the plurality of markers 340 is visible. The at least one aperture 352 may be configured to be smaller than or have a different shape from the outer perimeter of the patella-engaging member 310 so that the patella-engaging member 310 does not become accidentally uncoupled from the subject's patella. As shown, the coupling 350 may include a reinforced area (i.e., a generally annular portion proximate the at least one aperture 352) that helps urge the patella-engaging member 310 into engagement with the patella without having to indiscriminately tighten the coupling 350. In other embodiments the coupling 350 may alternatively be attached (e.g., by way of gluing or mechanical fasteners such as staples, screws, etc.) to one or more portions of the patella-engaging member 310 (e.g., the medial and lateral sides) so that the coupling 350 does not substantially cover the anterior side of the patella-engaging member 310.

The coupling 350 may comprise various materials known in the art such as natural or synthetic fabric, elastic, foam, etc. In one embodiment, the coupling 350 may be a knitted blend of nylon and Lycra® with an elastomeric foam inner layer (e.g., SuperWrap available from Fabrifoam® Products of Exton, Penn.) so that the coupling 350 allows for a high degree of multidirectional elasticity with adjustable compression and support. In this way the elastomeric foam inner layer increases purchase of the coupling 350 on the subject's skin and patella-engaging member 310 to make the coupling 350 and the patella-engaging member 310 non-migratory during knee flexion/extension without excess compression. In some embodiments, the patella-engaging member 310 may further include cushioning, such as, for example a thin layer of elastomeric foam, on its posterior surface to improve the comfort of the subject. The coupling 350 may include various fasteners (e.g., ties, buckles, snaps, buttons, hook-and-loop, etc.) to secure and adjust the placement and compression of the coupling 350 on the subject's knee. Furthermore, because the skin in front of (i.e., anterior) the patella and the patella itself do not move synchronously or at precisely the same time, pattern or direction (as is best evidenced during squatting and walking activities), it is preferred to tighten the coupling 350 enough to overcome skin movement in the patella region of the subject's knee. In this way, skin of the subject's knee in the patella region becomes substantially clamped between the patella and the patella-engaging member 310 so that the patella-engaging member 310 accurately and synchronously moves with the subject's patella.

The markers 340 (and markers 412, 422 shown in FIG. 4) may be any suitable active or passive marker device known in the art including but not limited to: reflective markers; high-visibility lights such as white, red or other colored LEDs; infrared light emitting diodes (LEDs), etc. After tracking the markers 340 with the first measuring portion 400b, the markers 340 may be removed from the member 310 and reattached, for example for employing the member 310 for subsequent diagnosis. To facilitate removal and reattachment of the markers 34 from the member 310, the markers 340 may be attached to a triad or other suitable member known in the art. As can be appreciated, the type of markers 340, 412 and 422 that are selected for the system 200 should be complementary or cooperative with the first measuring portion 400b. In one example system 200 where the first measuring portion 400b includes the OptoTrak® 3020 (available from Northern Digital, Inc. of Waterloo, Canada), it is preferred for the markers 340, 412, 422 to be infrared LEDs. In some embodiments where the markers 340, 412, 422 are active, they may be electrically connected to a portion of the motion analysis system 400, for example, the computer 400a or the first measuring portion 400b. As shown in FIG. 5, three markers 340 are provided and configured on the patella-engaging member 310 to have a nonlinear, triangular orientation. It is preferred that at least three markers 340 be provided and configured on the member 310 so that six degrees of freedom (DOF) tracking and motion analysis of a subject's patella can be performed. More than three markers 340 may be provided so long as the markers 340 are configured to have a nonlinear orientation. Although adjacent markers 340 may be spaced apart from each other by a minimum predetermined distance such as 15 mm, to facilitate greatest accuracy of tracking the markers 340 by the first measuring portion 400b, it is preferable for the markers 340 to be spaced apart as far as possible. Similarly, to facilitate tracking of the femoral and tibial portions 410, 420 of the second measuring portion 400c, it is preferable that the first and second plurality of markers 412, 422 each include at least three markers that are separated from each other by a minimum predetermined distance, which may be the same or different than the minimum predetermined distance of markers 340.

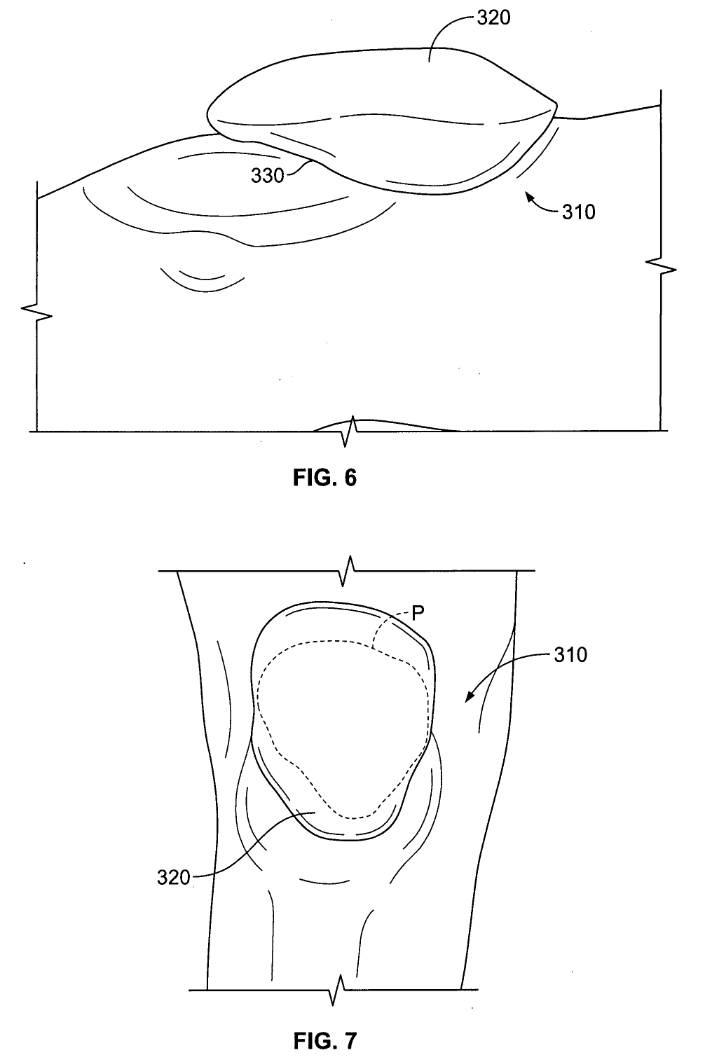

Referring now to FIGS. 6 and 7 the patella-engaging member 310 is described. As shown in FIG. 6, the patella-engaging member 310 includes a front or anterior surface 320 and a rear or posterior surface 330. As can be appreciated from FIGS. 4 and 5, the markers 340 are configured for attachment on and removal from the anterior surface 320 of the patella-engaging member 310. In some embodiments, the markers 340 may be attached to a triad or other suitable member known in the art. The patella-engaging member 310 is molded or formed so that the posterior surface 330 is custom-contoured and fitted/conformed to the subject's knee in the patella region so that the member 310 accurately and synchronously moves with the subject's patella when the coupling 350 is sufficiently tightened to overcome skin movement and soft tissue loading that are known to occur in the patella region during functional activities such as squatting and walking.

The patella-engaging member 310 may be made of a material that is custom molded/formed to a subject's knee in the patella region. In some embodiments the patella-engaging member 310 may be a plastic such as a thermoplastic material or resin. Some example materials include, but are not limited to: thermoplastic splinting materials such as the Aquaplast®, Ezeform®, Polyform®, Polyflex II® products that are available from Patterson Medical Products, Inc. of Bolingbrook, Ill. In other embodiments of the system 200 that are adapted and employed for diagnosing and treating maltracking and malalignment relative to another joint of interest, the subject-engaging member is custom molded/formed to that joint. Of course, when the system 200 is adapted and employed for diagnosing and treating maltracking and malalignment relative to another joint of interest, the coupling 350 is also adapted accordingly. For example, when the system 200 is adapted for diagnosing maltracking and malalignment of the scapula, the subject-engaging member is custom molded/formed to portions of the subject's shoulder and/or back and the coupling 350 may be in the form of a sling or the like.

The patella-engaging member 310 may be made from a “blank” (e.g., a sheet or block member) of thermoplastic material such as foam, plastic or resin that is custom formed, molded or otherwise configured to a subject of interest. One embodiment of the patella-engaging member 310 may be made by an example process comprising the steps of: heating the thermoplastic member to a predetermined softening temperature; orienting the softened thermoplastic member over the subject's patella, on the subject's knee; pressing the softened thermoplastic member on the subject's knee to deform the posterior side of the thermoplastic member; and removing and curing the deformed, softened thermoplastic member by, for example cooling it until the formed/molded patella-engaging member 310 is rigid. In some instances, the sheet or block member of thermoplastic material may be heated to the softening temperature by dipping or immersing the same in a bath of heated (e.g., boiling) liquid (e.g., water) for a predetermined amount of time or until the material becomes soft and malleable. As shown in FIG. 7, in some embodiments the patella-engaging member 310 may be formed so that the patella-engaging member 310 has a perimeter that slightly overhangs (i.e., extends past) a perimeter of the subject's patella P, which is shown by the dashed line. In this way, the patella-engaging member 310 may be wrapped around the edges of the patella P to grasp the patella P so that synchronous movement of the patella P and patella-engaging member 310 is enhanced.

Indeed, other embodiments of the patella-engaging member 310 may be made by other processes known in the art such as: photographing or scanning a subject's knee in three dimensions (e.g., with a GIS laser scanner, etc.) to develop a 3D model of the subject's knee and a patella-engaging member 310 that is customized thereto; and using one or more of injection/blow-molding equipment, CNC equipment, 3D printing and rapid prototyping (RP) equipment to provide the member 310 according to the model. Once the patella-engaging member 310 is custom molded/formed to a subject's knee, the patella-engaging apparatus 300 may be employed with the motion analysis system 400 for diagnosing patellar maltracking and malalignment. One example method for in vivo, noninvasive diagnosis of patellar maltracking and malalignment comprises the steps of: custom forming/molding a patella-engaging member to a subject's knee in the patella region; orienting the patella-engaging member 310 on the subject's knee, which is in full extension, over the patella; configuring markers 340 on the anterior side of the patella-engaging member 310; fitting the coupling 350 to the subject's knee to secure the patella-engaging member 310 to the subject's patella such that the markers 340 are visible (e.g., through the at least one aperture 352 shown in FIG. 5) and knee skin of the subject is sufficiently clamped between the member 310 and the subject's patella; and tracking and analyzing movement of the markers 340 with the motion analysis system 400 to diagnose maltracking and malalignment of the subject's patella.

The following example further illustrates the invention but, of course, should not be construed as in any way limiting its scope.

### EXAMPLE 1

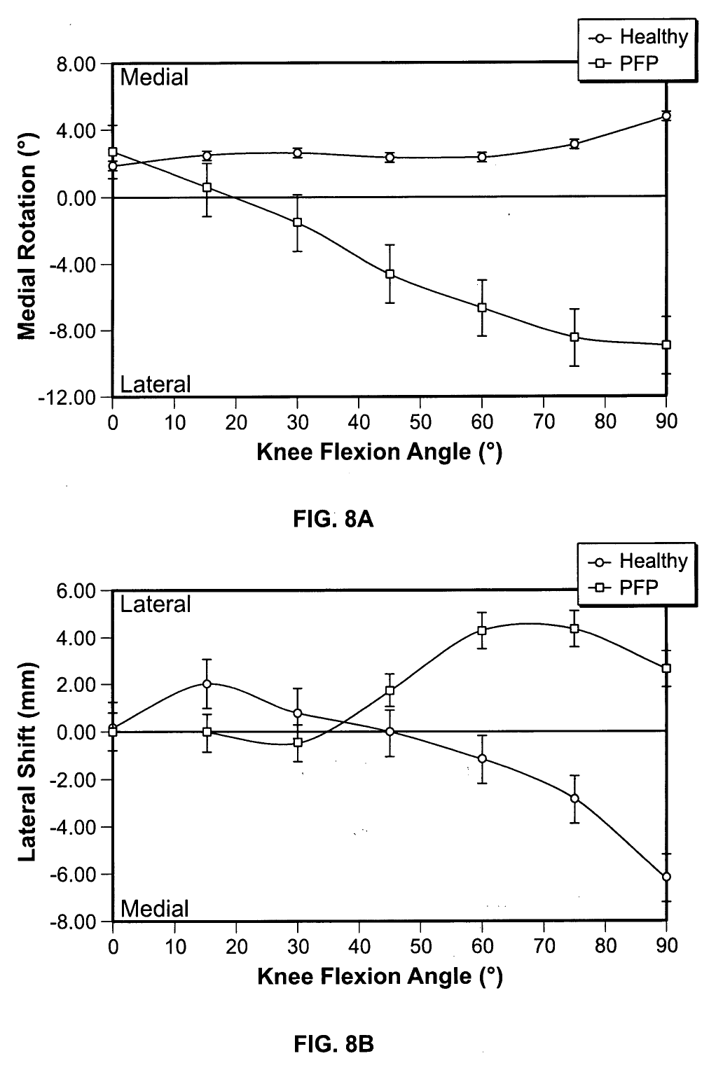

Using the OptoTrak® 3020 (available from Northern Digital, Inc. of Waterloo, Canada) for digitization and tracking measurements, three-dimensional patellar kinematics was assessed in 8 healthy volunteers (TABLE 1-HS) and 5 volunteers with PFP syndrome (TABLE 1-PFP). After each subject was custom-fitted with the foregoing-described patella-engaging apparatus 300, subjects were asked to stand upright with feet shoulder-width apart while bony landmarks surrounding the knee were digitized. Then, each subject was asked to perform a series of squat maneuvers to self-selected depth (>90°) while patellofemoral kinematics were recorded. Results are summarized in the following table and illustrated in FIGS. 8a and 8b.

TABLE 1

| | | |
|---|---|---|
| | Medial | |
| Medial | Rotation | Lateral |
| Tilt (°) | (°) | Shift (mm) |

| KFA | HS | PFP | HS | PFP | HS | PFP |
|---|---|---|---|---|---|---|
| 0° | 0.37 | 0.31 | 1.87 | 2.63 | 0.25 | −0.02 |
| 15° | 2.18 | 1.50 | 2.43 | 0.58 | 2.00 | −0.02 |
| 30° | 3.32 | 2.30 | 2.64 | −1.57 | 0.78 | −0.44 |
| 45° | 3.52 | 3.13 | 2.34 | −4.62 | 0.01 | 1.68 |
| 60° | 3.08 | 3.89 | 2.32 | −6.71 | −1.17 | 4.27 |
| 75° | 2.51 | 3.66 | 3.09 | −8.51 | −2.83 | 4.32 |
| 90° | −0.66 | 2.58 | 4.77 | −9.14 | −6.14 | 2.63 |

| | | | |
|---|---|---|---|
| p-value | 0.290 | 0.001 | 0.025 |

The average self-selected squat depth for control subjects was 102.57° (16.38) knee flexion. The average self-selected squat depth for PFP subjects was 104.70° (20.37) knee flexion. All subjects squatted to at least 90° knee flexion. For both PFP and healthy subjects, patellar flexion lagged behind knee flexion angle. Referring to TABLE 1 and FIGS. 8a and 8b, during knee flexion from 0-90°, the patella flexed continuously from 0-33°. For healthy subjects, the patella gradually tilted medially as the knee was flexed to 45°, and then gradually tilted laterally as flexion continued to 90°. For PFP subjects, the patella tilted medially as the knee was flexed to 60°, then tilted laterally as flexion continued to 90°. For healthy subjects, the patella rotated medially, from 2° and gradually rotated medially to 5° as the knee was flexed to 90°. For PFP subjects, the patella rotated laterally from 3° at full extension to 9° at 90°. For healthy subjects, the patella gradually shifted medially (8 mm) as the knee was flexed from 15° to 90°. For PFP subjects, the patella shifted laterally (4 mm) as the knee was flexed from 30° to 75°, then shifted medially (2°) as the knee continued to flex to 90°. In subjects with PFP, the patella rotated significantly more laterally than in healthy subjects. In PFP subjects the patella underwent significantly more lateral translation than in healthy subjects.

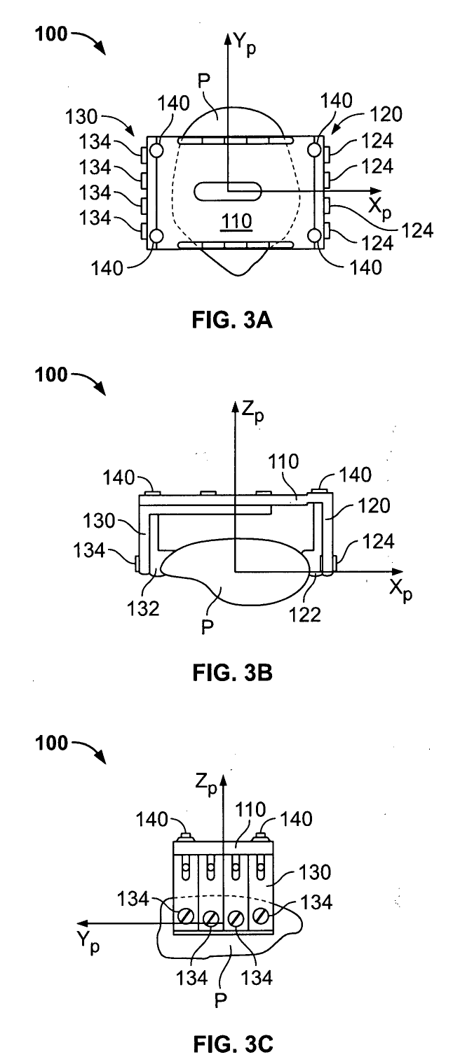

To validate the experimental in vivo, noninvasive results, two fresh-frozen cadaver specimens were used to corroborate patellar tracking measurements. In addition to the patella-engaging apparatus 300 and the motion analysis system 400 including second measuring portion 400c (e.g., a goniometer), cortical screws were inserted into the patella, femur, and tibia. A small Y-shaped frame (triad) with markers on each of the three arms was attached to each of the cortical screws (as shown in FIG. 3 of the Lin et al. reference, which is incorporated herein by reference). Patello- and tibiofemoral movements were induced using a servomotor to move the knee passively through a simulated squat (0° to 120°) with the major muscles crossing the knee loaded according to their physiological cross sectional areas. Patellofemoral movements tracked by motion analysis system 400 according to the markers attached to the triads and by the apparatus 300 and goniometer were compared in 6 degrees of freedom (DOF). Results from validation experiments showed that for a range of knee flexion (0° to 100°), the differences in displacements between the patella and the markers on the apparatus 300 were less than ±1.2° for rotations and ±0.5 mm for translations.

Therefore, while error due to skin movement cannot be eliminated completely, it is possible to substantially minimize it by way of the patella-engaging apparatus 300, which is shown and described herein, to perform noninvasive measurements of patellar tracking in vitro with an accuracy that is an order of magnitude better than the smallest change measured. In this example, patellofemoral kinematics from healthy subjects closely match tracking patterns previously reported and significant differences in tracking patterns can be seen. Furthermore, the experimental results are consistent with the view that abnormal patellar tracking is closely related to PFP syndrome.

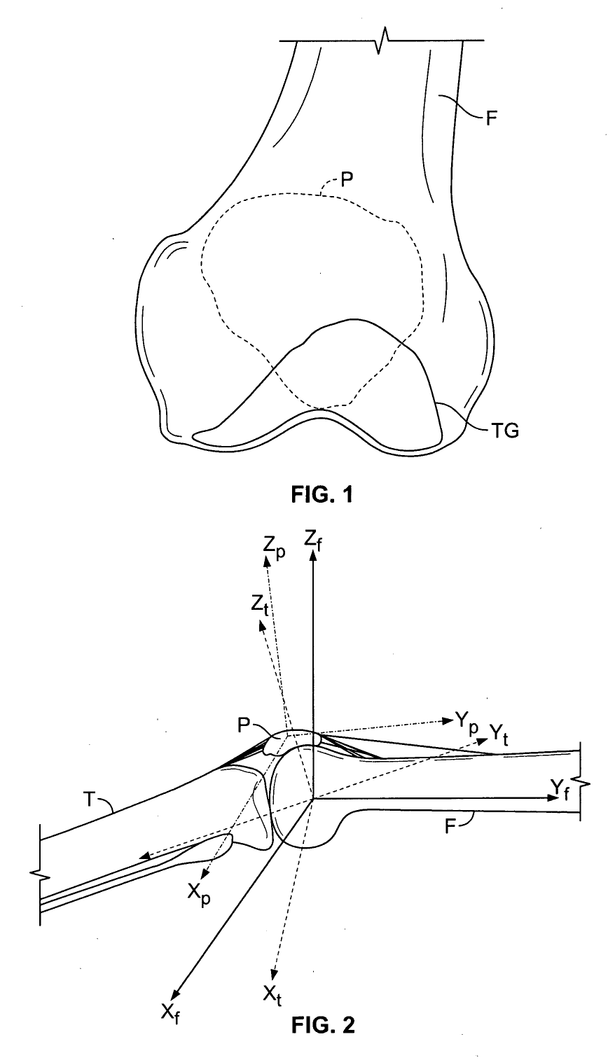

Because the patella-engaging member 310 is custom-fit to a subject's knee in the patella region, the patella-engaging member 310 may be employed in a nonoperative treatment method and system for correcting patellar malalignment and maltracking. In a treatment system and method the patella-engaging member 310 can be used to engage the patella for helping to move the patella into a desired position and orientation (e.g., aligned with the trochlear groove TG shown in FIG. 1) to correct the malalignment and maltracking. After the patella is moved with the patella-engaging member 310, an alignment-maintaining member is coupled with the patella-engaging member 310 and the subject's knee for supporting the subject's patella in the realigned position and orientation. In some embodiments the alignment-maintaining member may comprise the coupling 350, however, in other embodiments the alignment-maintaining member may comprise tape or an orthotic device such as, for example, a knee brace or wrap.

As disclosed in Chapter 12 of “Disorders of the Patellofemoral Joint,” Fourth edition by Fulkerson, “Application of tape to the skin around the patellofemoral joint undoubtedly moves skin more than patella.” Thus, as can be appreciated, by using the patella-engaging member 310 in systems and methods for nonoperative treatment, the patella-engaging member 310 provides a means to: substantially overcome interference due to skin and soft tissue loading that makes it difficult to move and properly position and orient or align the patella. In this way, the effectiveness of taping techniques (e.g., the McConnell Taping Technique) and orthotic devices (e.g., braces and wraps such as the Tru-Pull system available from DJO Incorporated of Vista, Calif.) can be improved. For example, in a brace or wrap that includes a patellar opening, the patella-engaging member 310 may be sewn, glued or otherwise connected to a portion of the brace or wrap proximate to the patellar opening so that, when the brace or wrap is fitted to a subject, the member 310 moves the patella to a desired position and orientation and maintains the patella in that desired position and orientation.

One example treatment method comprises the steps of: configuring a patella-engaging member to have a posterior surface that is custom-contoured to the knee of a subject in the patella region; engaging the patella-engaging member with the subject's patella; moving the patella-engaging member to position the patella in a desired orientation; and taping the patella-engaging member to the knee of the subject to maintain the patella in the desired position and orientation. Another example treatment method comprises the steps of: configuring a patella-engaging member to have a posterior surface that is custom-contoured to the knee of a subject in the patella region; attaching the patella-engaging member to a patellar portion of an orthotic device; and fitting the orthotic device to the knee of the subject so that the patella-engaging member moves the patella to a desired position and orientation and maintains the patella in the desired position and orientation.

In some instances, the patella-engaging member 310 may be used in the foregoing-described system and method for diagnosing patellar maltracking and malalignment, after which the patella-engaging member 310 is reused (e.g., without the plurality of markers 340), and subsequently employed in the nonoperative treatment method and system. To this end, one example treatment method comprises the steps of: diagnosing malalignment and maltracking of the subject's patella using the patella-engaging member; and relative to the diagnosing step, taping or bracing the custom-formed patella-engaging member to the subject's knee for correcting the malalignment and maltracking. However, it should be appreciated that the patella-engaging member 310 may be configured specifically for use in nonoperative treatment applications and is not limited to being first employed in a diagnostic system and method.

In another aspect the system 200 may facilitate a method for evaluating the effectiveness of treatment of patellar malalignment and maltracking since the foregoing-described patella-engaging apparatus 300 provides accurate and repeatable diagnosis of patellar maltracking and malalignment. An example method comprises the steps of: a) determining a pre-treatment amount of patellar malalignment and maltracking in vivo and noninvasively; b) treating the determined malalignment and maltracking; c) determining a post-treatment amount of patellar malalignment and maltracking in vivo and noninvasively subsequent to the treating step; and d) comparing the pre-treatment and post-treatment amounts of patellar malalignment and maltracking. Determining step a) may include the steps of custom-configuring a patella-engaging member 310 relative to a knee of a subject in the patella region; configuring a plurality of markers 340 on an anterior side of the patella-engaging member310; coupling the patella-engaging member 310 to the knee of the subject (e.g., with coupling 350); and tracking movement of the plurality of markers 340 in three dimensions during a functional activity being performed by the subject.

The treating step b) may include one or more steps including, but not limited to: strengthening muscle(s) of the subject's leg; performing an appropriate surgical procedure on the subject's leg to improve functionality of the patella; and nonoperative treatment such as taping the subject's knee and/or fitting the subject with one or more orthotic devices such as knee braces, shoe inserts, etc. In some instances the patella-engaging member 310 may be used in the treating step b) in combination with tape or an orthotic device by, for example, removing the plurality of markers 340 from the patella-engaging member 310. If the plurality of markers 340 were removed for employing the patella-engaging member 310 during the treating step b), the markers 340 may be subsequently reattached to the patella-engaging member 310 before or during the determining step c).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Various embodiments of this invention are described herein. It should be understood that the illustrated embodiments are exemplary only, and should not be taken as limiting the scope of the invention. Furthermore, although the present invention is described in the context of use for diagnosing patellar maltracking and malalignment, the described system and method may be adapted and employed for diagnosing maltracking and malalignment in other joints, for example the scapula/shoulder blade as mentioned briefly above as well as other human and animal joints.

1. An in vivo, noninvasive system for diagnosing patellar maltracking and malalignment of a knee of a subject during a functional activity including at least one of a first knee movement from frill extension to 90° flexion and a second knee movement from 90° flexion to frill extension, comprising:
a member that includes an anterior surface and a posterior surface custom-contoured to the knee of the subject;
a plurality of markers configured on the anterior surface;
a coupling adapted to engage the member with the patella of the subject, the coupling causing the member and the patella of the subject to move synchronously during the first and second knee movements; and
a motion analysis system that includes an optical portion for tracking movement of the plurality of markers in three-dimensions during the functional activity including at least one of the first knee movement and the second knee movement.

2. The system of claim 1 wherein the coupling comprises an elastic member that is configured to wrap around the knee of the subject, the elastic member including at least one aperture through which the plurality of markers is visible.

3. The system of claim 2 wherein the elastic member is tensioned to overcome skin movement and soft tissue loading that occur in the patella region of the knee of the subject.

4. The system of claim 2 wherein the coupling further comprises an inner layer that increases purchase of the elastic member on the knee.

5. The system of claim 4 wherein the inner layer comprises elastomeric foam.

6. The system of claim 1 wherein the member comprises a thermoplastic material that is deformable above a predetermined softening temperature.

7. The system of claim 1 wherein the member comprises a blank that is machined or molded.

8. The system of claim 1 wherein the patella of the subject has an outer perimeter and the member is configured to have a perimeter that overlaps the outer perimeter of the patella.

9. The system of claim 1 wherein the plurality of markers comprise at least three markers, the at least three markers being configured in a nonlinear orientation.

10. The system of claim 1 wherein the markers are chosen from the group consisting of active markers and passive markers.

11. The system of claim 10 wherein each marker is active and comprises an LED.

12. The system of claim 10 wherein each marker is passive and comprises a reflective member.

13. The system of claim 1 wherein the motion analysis system further includes a second portion that is configured to measure relative movement of the tibia and the femur of the subject during the functional activity.

14. The system of claim 13 wherein the second portion comprises a goniometer.

15. The system of claim 13 wherein the second portion of the motion analysis system further comprises a second plurality of markers, wherein the optical portion tracks movement of the second plurality of markers in three-dimensions during the functional activity.

16. The system of claim 15 wherein the second plurality of markers comprises:
at least three markers configured nonlinearly on a tibial part of the second portion; and
at least three markers configured nonlinearly on a femoral part of the second portion.

17-54. (canceled)

55. An in vivo, noninvasive system for diagnosing patellar maltracking and malalignment of a knee of a subject during a functional activity including at least one of a first knee movement from frill extension to 90° flexion and a second knee movement from 90° flexion to frill extension, comprising:
a member that includes an anterior surface and a posterior surface custom-contoured to the knee of the subject;
an elastic member configured to wrap around the knee of the subject for urging the member against the knee of the subject, the elastic member being tensioned to clamp skin and soft tissue between the member and the patella for overcoming movement of skin and loading of soft tissue that occur during the first and second knee movements;
a plurality of markers configured on the anterior surface, the plurality of markers being viewable through at least one aperture of the elastic member; and
a motion analysis system that includes an optical portion for tracking movement of the plurality of markers in three-dimensions during the first and second knee movements of the functional activity.

56. The system of claim 55 wherein the elastic member further comprises an inner layer that increases purchase of the elastic member on the knee.

57. The system of claim 56 wherein the inner layer comprises elastomeric foam.

58. The system of claim 55 wherein the member comprises a thermoplastic material that is deformable above a predetermined softening temperature.

59. The system of claim 55 wherein the member comprises a blank that is machined or molded.

60. The system of claim 55 wherein the patella of the subject has an outer perimeter and the member is configured to have a perimeter that overlaps the outer perimeter of the patella.

61. The system of claim 55 wherein the plurality of markers comprise at least three markers, the at least three markers being configured in a nonlinear orientation.

62. The system of claim 55 wherein the markers are chosen from the group consisting of active markers and passive markers.

63. The system of claim 62 wherein each marker is active and comprises an LED.

64. The system of claim 62 wherein each marker is passive and comprises a reflective member.

65. The system of claim 55 wherein the motion analysis system further includes a second portion that is configured to measure relative movement of the tibia and the femur of the subject during the functional activity.

66. The system of claim 65 wherein the second portion comprises a goniometer.

67. The system of claim 65 wherein the second portion of the motion analysis system further comprises a second plurality of markers, wherein the optical portion tracks movement of the second plurality of markers in three-dimensions during the functional activity.

68. The system of claim 67 wherein the second plurality of markers comprises:
at least three markers configured nonlinearly on a tibial part of the second portion; and
at least three markers configured nonlinearly on a femoral part of the second portion.
